(19) United States
(12) Patent Application Publication
(10) Pub. No.: US 2016/0174476 A1
(43) Pub. Date: Jun. 23, 2016

(54) ALGAE GROWTH USING PERISTALTIC PUMP

(71) Applicant: Marsh Allen, Gig Harbor, WA (US)

(72) Inventor: Marsh Allen, Gig Harbor, WA (US)

(21) Appl. No.: 14/971,864

(22) Filed: Dec. 16, 2015

Related U.S. Application Data
(60) Provisional application No. 62/093,304, filed on Dec. 17, 2014.

(51) Int. Cl. A01G 25/16; C12M 1/00; A01G 1/00

(52) U.S. Cl. CPC A01G 25/16; A01G 1/001; C12M 21/02; C12M 31/08; C12M 29/18; C12M 29/00

(57) ABSTRACT

The present disclosure generally relates to peristaltic pump systems, and methods of using peristaltic pump systems to grow algae. In some implementations, a peristaltic pump system includes a frame supporting a plurality of rollers powered by a motor, and the rollers can be actuated to pump a fluid containing algae through an elongate, looped tube. In some implementations, such a system includes a ball trap assembly that can allow balls to be inserted into and to be removed from the tube, and that can synchronize the passage of balls through the tube with movement of the rollers. In some implementations, such a system includes a harvesting system that can be used to harvest algae from the system.

### BACKGROUND

1. Technical Field

The present disclosure generally relates to peristaltic pump systems and methods of using peristaltic pump systems to grow algae.

2. Description of the Related Art

Algae has various uses. For example, algae can be used to convert carbon dioxide into long chain hydrocarbons such as starches or oils. As another example, some algae can be used as dietary supplements. Systems for growing algae and corresponding methods of growing algae are known but suffer from various drawbacks. For example, some algae growth systems rely on pumping systems such as centrifugal pumping systems, which can destroy the algae. Further, some algae growth systems require large amounts of water, and are thus not suitable for use in low-water, dry environments.

### BRIEF SUMMARY

A system may be summarized as including: a looped tube that holds water, algae, and a ball; a peristaltic pump positioned to pump the water, the algae, and the ball through the looped tube; and a synchronizer coupled to synchronize pumping action of the peristaltic pump with movement of the ball through the looped tube.

The looped tube may lie within a horizontal plane or on a dried lake bed. The ball may be a first ball that is heavier than water and the looped tube may hold a second ball that is lighter than water. The ball may have a diameter that is within two inches of an inside diameter of the looped tube. The system may further include a reflective tarp positioned under the looped tube, a vertical exhaust pipe having a bottom end coupled to the looped tube and a top end coupled to a relief valve, or an input valve including a sintered metal.

A method may be summarized as including: positioning water, algae, and a ball within a looped tube; driving a roller of a peristaltic pump to compress the looped tube; translating the roller across the looped tube, thereby pumping the water, the algae, and the ball through the looped tube; and synchronizing the driving of the roller of the peristaltic pump to compress the looped tube with a passage of the ball through the looped tube.

The positioning the ball within the looped tube may include closing a valve between a housing and the looped tube; positioning the ball within the housing; sealing the housing; opening the valve; and moving the ball from the housing into the looped tube. The method may further include removing the ball from the looped tube, the removing comprising: retaining the ball in place within the looped tube; opening a valve between a housing and the looped tube; moving the ball from the looped tube into the housing; closing the valve; opening the housing; and removing the ball from the housing.

A system may be summarized as including: a looped tube that has a passage having a first diameter to contain water, algae, and a ball having a second diameter; and a valve that has an orifice fluidically coupled to the looped tube, wherein the orifice has a third diameter greater than the second diameter such that a difference between the first diameter and the third diameter is less than two inches.

A method may be summarized as including: using a peristaltic pump to pump water and algae through a looped tube; drawing a portion of the water and algae out of the looped tube; positioning a filter on a perforated plate; pouring the portion of the water and algae onto a first surface of the filter; and reducing an air pressure on a second surface of the filter opposite the first surface to draw the water through the filter.

Drawing the portion of the water and algae out of the looped tube may include drawing the portion of the water and algae into a holding tank. Pouring the portion of the water and algae onto the first surface of the filter may include pouring the first portion of the water and algae from the holding tank. The method may further include measuring a fluid pressure within the looped tube by observing a water level in a vertical portion of a pipe coupled to the looped tube, or, when the fluid pressure within the looped tube exceeds a threshold fluid pressure, relieving the fluid pressure within the looped tube by allowing the water and algae to flow from the looped tube, through the vertical portion of the pipe, into the holding tank.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not necessarily drawn to scale, and some of these elements may have been arbitrarily enlarged and positioned to improve drawing legibility. Further, the particular shapes of the elements, as drawn, are not intended to convey any information regarding required shapes of the particular elements, and may have been selected for ease of recognition in the drawings.

FIG. 1 illustrates an algae growth system, according to at least one illustrated embodiment.

FIG. 2 illustrates a portion of a tube of an algae growth system, according to at least one illustrated embodiment.

FIG. 3 illustrates a portion of a tube of an algae growth system, according to at least one illustrated embodiment.

FIG. 4 illustrates a corner of a tube of an algae growth system, according to at least one illustrated embodiment.

FIG. 5 illustrates a relief valve of an algae growth system, according to at least one illustrated embodiment.

FIG. 6 illustrates a corner of a tube of an algae growth system, according to at least one illustrated embodiment.

FIG. 7 illustrates additional details of the corner of the tube of the algae growth system of FIG. 6, according to at least one illustrated embodiment.

FIG. 8 illustrates additional details of the corner of the tube of the algae growth system of FIGS. 6 and 7, according to at least one illustrated embodiment.

FIG. 9 illustrates a pump of an algae growth system, according to at least one illustrated embodiment.

FIG. 10 illustrates additional details of the pump of the algae growth system of FIG. 9, according to at least one illustrated embodiment.

FIG. 11 illustrates additional details of the pump of the algae growth system of FIGS. 9 and 10, according to at least one illustrated embodiment.

FIG. 12 illustrates a ball passing through a tube of an algae growth system, according to at least one illustrated embodiment.

FIG. 13 illustrates the ball passing through the tube of the algae growth system of FIG. 12, according to at least one illustrated embodiment.

FIG. 14 illustrates a ball trap of an algae growth system, according to at least one illustrated embodiment.

FIG. 15 illustrates additional details of the ball trap of the algae growth system of FIG. 14, according to at least one illustrated embodiment.

FIG. 16 illustrates additional details of the ball trap of the algae growth system of FIGS. 14 and 15, according to at least one illustrated embodiment.

FIG. 17 illustrates an automated switch of an algae growth system, according to at least one illustrated embodiment.

FIG. 18 illustrates additional details of the automated switch of the algae growth system of FIG. 17, according to at least one illustrated embodiment.

FIG. 19 illustrates a pipe and a holding tank of an algae growth system, according to at least one illustrated embodiment.

FIG. 20 illustrates the pipe and the holding tank of the algae growth system of FIG. 19, according to at least one illustrated embodiment.

FIG. 21 illustrates the pipe and the holding tank of the algae growth system of FIGS. 19 and 20, according to at least one illustrated embodiment.

FIG. 22 illustrates a harvesting apparatus of an algae growth system, according to at least one illustrated embodiment.

FIG. 23 illustrates additional details of the harvesting apparatus of the algae growth system of FIG. 22, according to at least one illustrated embodiment.

FIG. 24 illustrates additional details of the harvesting apparatus of the algae growth system of FIGS. 22 and 23, according to at least one illustrated embodiment.

FIG. 25 illustrates additional details of the harvesting apparatus of the algae growth system of FIGS. 22-24, according to at least one illustrated embodiment.

FIG. 26 illustrates a vertical pipe of an algae growth system, according to at least one illustrated embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with the technology have not been shown or described in detail to avoid unnecessarily obscuring descriptions of the embodiments.

Unless the context requires otherwise, throughout the specification and claims that follow, the word “comprising” is synonymous with “including,” and is inclusive or open-ended (i.e., does not exclude additional, unrecited elements or method acts).

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the context clearly dictates otherwise. It should also be noted that the term “or” is generally employed in its broadest sense, that is, as meaning “and/or” unless the context clearly dictates otherwise.

The headings and Abstract of the Disclosure provided herein are for convenience only and do not limit the scope or meaning of the embodiments.

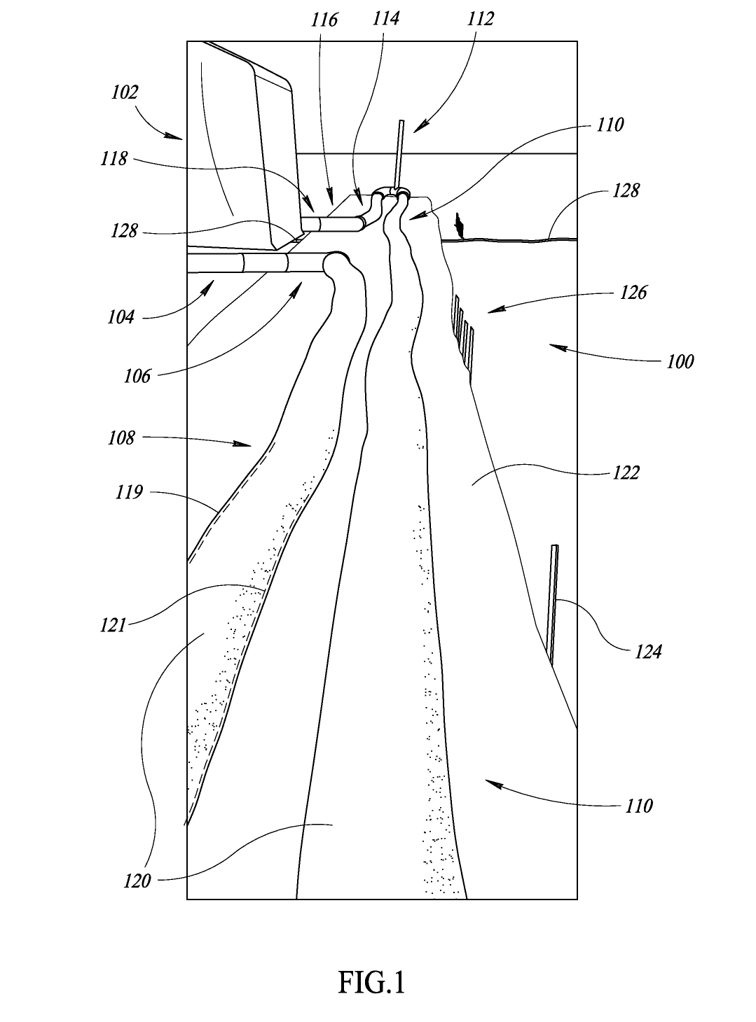

FIG. 1 shows an algae growth system 100. The system 100 includes a pump 200 and other equipment (not shown in FIG. 1 but described in greater detail below) located in a tent 102 or a pump room or pump house, and an elongated tube 120. The tube 120 can have an outer wall 119 that separates an inner passage 121 from an exterior of the tube 120. The pump 200 can pump a fluid through the tube 120, which can form an extended loop through which the fluid can repeatedly or continuously flow. In some implementations, the looped tube 120 can form a closed path through which the fluid can flow. In other implementations, the looped tube can form a selectively closed path, such that the path can be selectively opened and closed, such as at one or more valves to introduce items into, or remove items from, the tube 120.

For example, the pump 200 can pump the fluid out of the tent 102 into a first segment 104 of the tube 120, which can carry the fluid away from the tent 102 to a first corner 106 of the tube 120, which fluidically couples the first segment 104 to a second segment 108 of the tube 120. The tube 120 can turn a second corner 148 (not shown in FIG. 1 but described in greater detail below), which fluidically couples the second segment 108 to a third segment 110 of the tube 120, and a third corner 112 which fluidically couples the third segment 110 to a fourth segment 114 of the tube 120. The fourth segment 114 can carry the fluid to a fourth corner 116 of the tube 120, which fluidically couples the fourth segment 114 to a fifth segment 118 of the tube 120. The first segment 104 and the fifth segment 118 can be fluidically coupled to one another through the tent 102, as described in greater detail below.

While the tube 120 forms a loop having a specific shape, in alternative implementations, the tube 120 can form a loop having any desired shape. The tube 120 can form a loop having any number of corners connecting a corresponding number of segments. As examples, the tube 120 can form a loop having a square, rectangular, circular, triangular, or other geometric shape. In some implementations, the tube 120 can form a loop having a shape designed so that the tube 120 remains at a constant elevation, such as by following the contour lines of a topographical map. In some implementations, the tube 120 can form a loop having multiple, repeated undulations, such that a greater length of the tube takes up a relatively smaller surface area than otherwise. In some implementations, the tube 120 can form a loop having sub-loops or branches therefrom. For example, the tube 120 can have a shape generally resembling a ladder, such that fluid can flow outward from the pump 200 along a first main segment, in parallel across several connecting segments to a second main segment, and toward the pump 200 through the second main segment.

The tube 120 can be filled with a fluid, for example a liquid such as water, which the pump 200 can circulate through the tube 120 under pressure. The fluid can contain an algae material which can grow in the tube 120, together with nutrients and other supplemental materials, as desired. Tube 120 can be transparent or translucent, allowing light to enter the tube, for example, to drive algae photosynthesis and growth. Thus, the system 100 can be used to grow algae.

FIG. 1 also shows that the system 100 can include a tarp or other sheet of material 122 positioned underneath the tube 120. The sheet 122 can have a white or other relatively light color so that the sheet 122 reflects light (e.g., from the sun) into the tube 120, increasing the energy available for photosynthesis. The system 100 can also include a number of stakes, flags, or other signposts in the ground adjacent to the tube 120. For example, the system can include a first reference stake 124 spaced apart by a reference distance from a set of measurement stakes 126. The stakes 124, 126 can be used to measure the speed at which the fluid flows through the tube 120, as described in greater detail below. The system 100 can also include a hose 128 which can carry fluids to and from the tent 102, such as by carrying water from a water source to the tent 102. As shown in FIG. 1, the system 100 is capable of working and is designed to work on a relatively flat surface such as a flat or level ground surface. Thus, the tube 120 can lie in or substantially within a single plane, such as a single horizontal plane. Such an implementation can minimize the pressure differential in the fluid in different locations in the tube 120. In alternative implementations, however, the tube 120 need not lie in or substantially within a single plane.

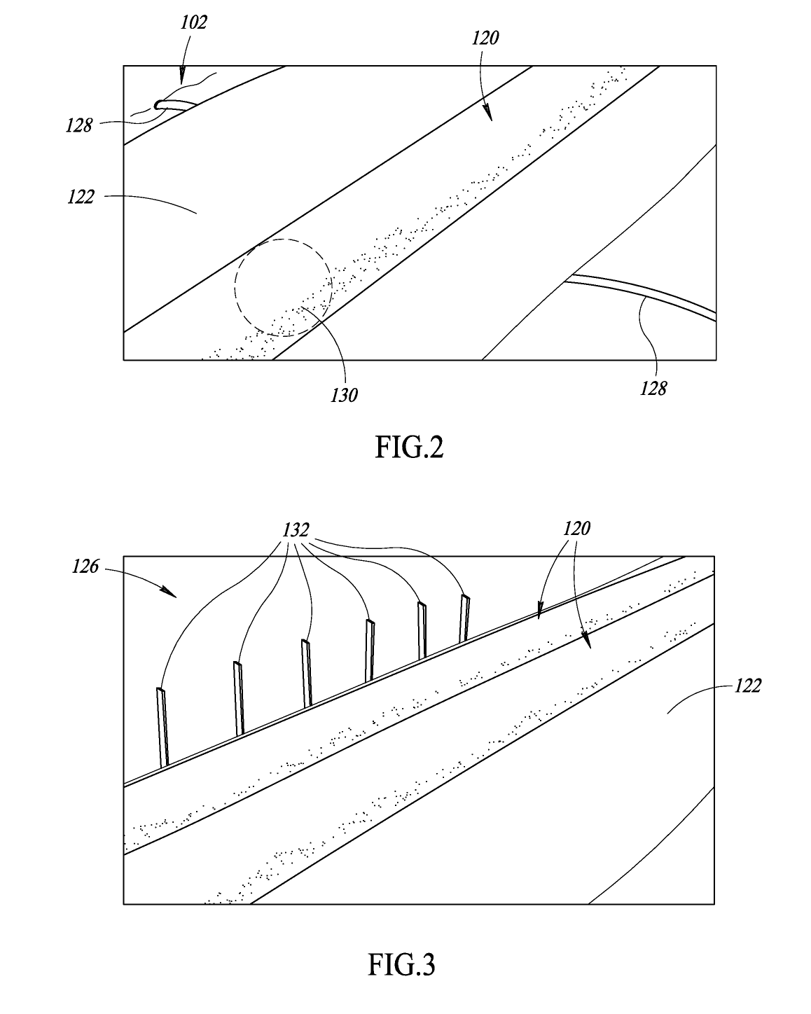

FIG. 2 shows that the system 100 can include one or more balls 130 selectively introducible into the passage 121 of the tube 120. The balls 130 can have a diameter approaching, but less than, an inside diameter of the tube 120. For example, a difference between the diameter of the balls 130 and the inside diameter of the tube 120 can be less than 2 inches, or less than 1 inch, or less than ½ inch, or less than ¼ inch. Thus, the fluid pressures in the tube 120 can cause the balls 130 to travel through the tube 120 with the fluid. In some cases, the balls 130 can be heavier than the fluid (e.g., the balls 130 can be filled with salt water), such that the balls 130 travel along the bottom of the tube 120, such as by rolling along the bottom of the tube 120. In other cases, the balls 130 can be lighter than water (e.g., the balls 130 can be filled partially or completely with air), such that the balls 130 travel along the top of the tube 120, such as by rolling along the top of the tube 120. The balls 130 can disturb the algae growing in the tube 120 and ensure that the algae moves with the fluid through the tube 120. The balls can also mix the fluid, algae, and any supplemental materials as they pass through the tube 120. In some implementations, the system 100 can include a plurality of balls 130 within the tube 120, with heavier-than-water balls 130 alternating with lighter-than-water balls 130, to further ensure increased mixing and transport of the algae and nutrients throughout the tube 120.

FIG. 3 shows that the set of measurement stakes 126 can include a plurality of individual measurement stakes 132, such as six individual measurement stakes. Each of the measurement stakes 132 can be positioned adjacent to the tube 120 at a predetermined distance from the reference stake 124. As one specific example, the six measurement stakes 132 shown in FIG. 3 can be positioned 25, 26, 27, 28, 29, and 30 feet from the reference stake 124. The stakes 124, 132, and one of the balls 130 can be used to determine the speed at which the fluid is being pumped through the tube 120. For example, a timer can be started when the ball 130 passes the reference stake 124, and a measurement taken when the ball 130 reaches one of the measurement stakes 132. The time taken and the distance traveled can be used to compute a speed of the fluid. In one specific implementation, the timer can be allowed to run until one minute has elapsed. At this time, the distance traveled (as indicated by the measurement stakes 132) can be used to compute the speed in a straightforward manner.

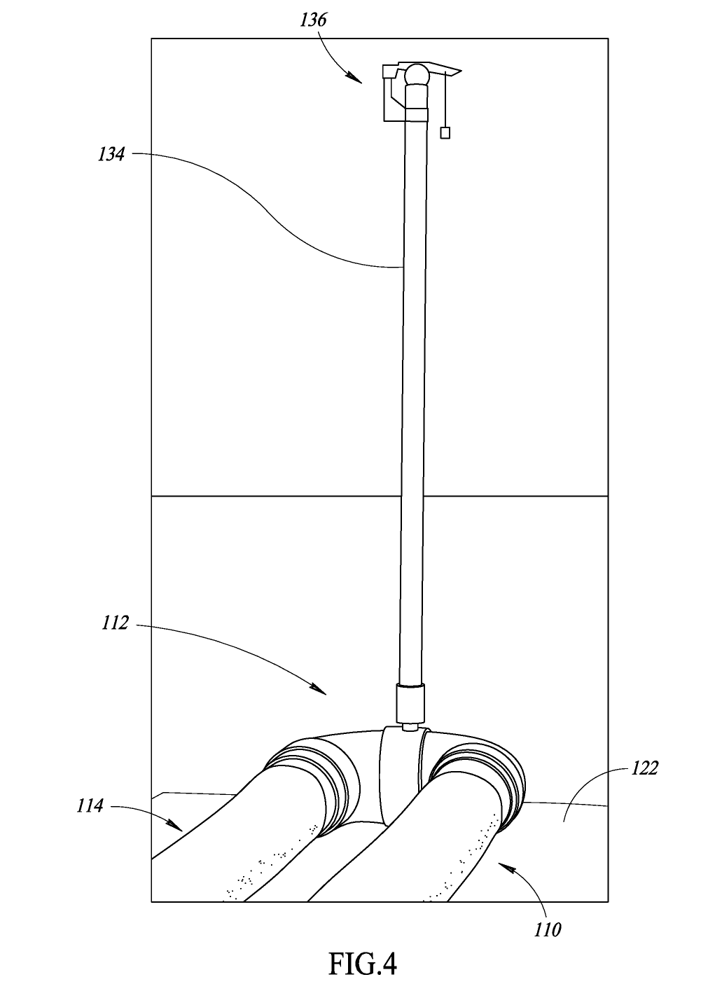

FIG. 4 shows that the third corner 112 of the tube 120 can be fluidically coupled to a bottom end of a vertical exhaust pipe 134, the top end of which can be physically coupled to a relief valve 136, which is shown in greater detail in FIG. 5. FIG. 5 shows that the relief valve 136 can include a ball 138 having a diameter larger than an inside diameter of the exhaust pipe 134, and positioned to rest on and block the open top end of the exhaust pipe 134. The relief valve 136 can also include a support element 140 physically coupled to the exhaust pipe 134, a lid 142 physically coupled to the support element 140 at a hinge 144 such that the lid is rotatable with respect to the support element 140, and a weight 146 physically coupled to the lid 142 at a location opposite the hinge 144. The lid 142 can be positioned on top of the ball 138 such that the weight 146 acts to keep the lid pressed against the ball 138 and thereby the ball 138 pressed against the open top end of the exhaust pipe 134.

The relief valve 136 can provide a plug which can prevent exhaust gasses (e.g., waste gasses produced by the algae growing in the tube 120) escaping from the system 100 and prevent debris from entering the system 100, such as by falling into the exhaust pipe 134. The relief valve 136 can also allow exhaust gasses to escape the system 100 once sufficient exhaust gas has been formed to overcome the weight 146 acting to keep the ball 138 pressed against the open top end of the exhaust pipe 134. The relief valve 136 is advantageous because it is passive, and allows excess exhaust gasses to escape without damage to the valve 136.

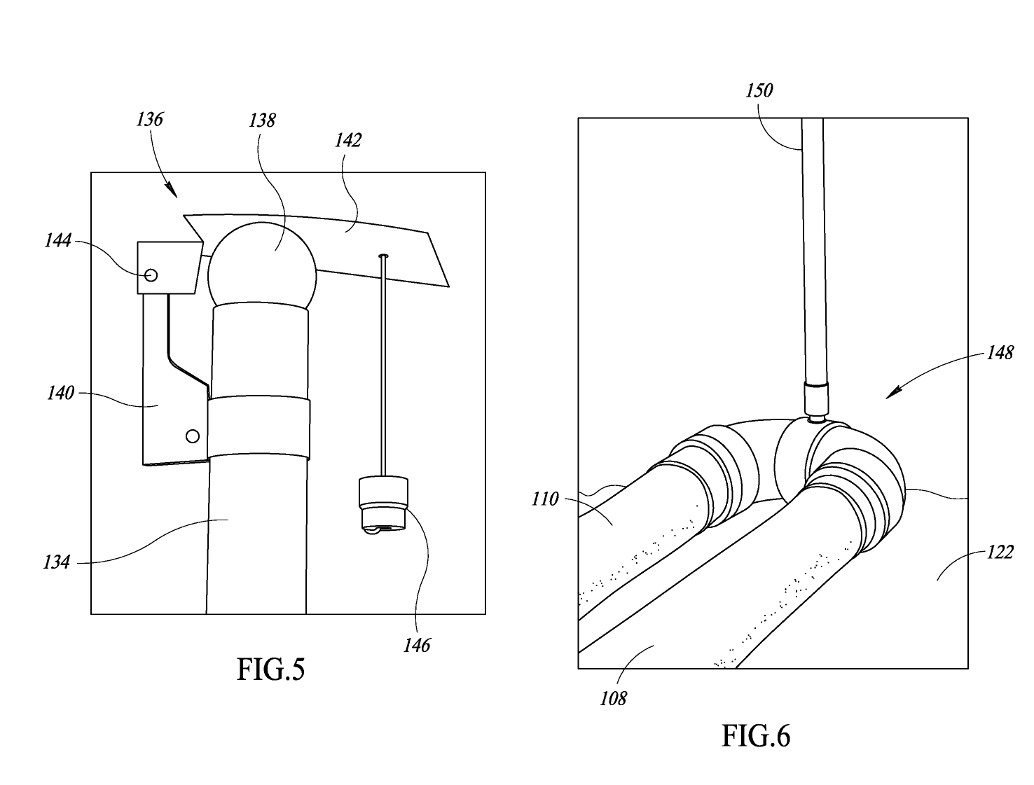

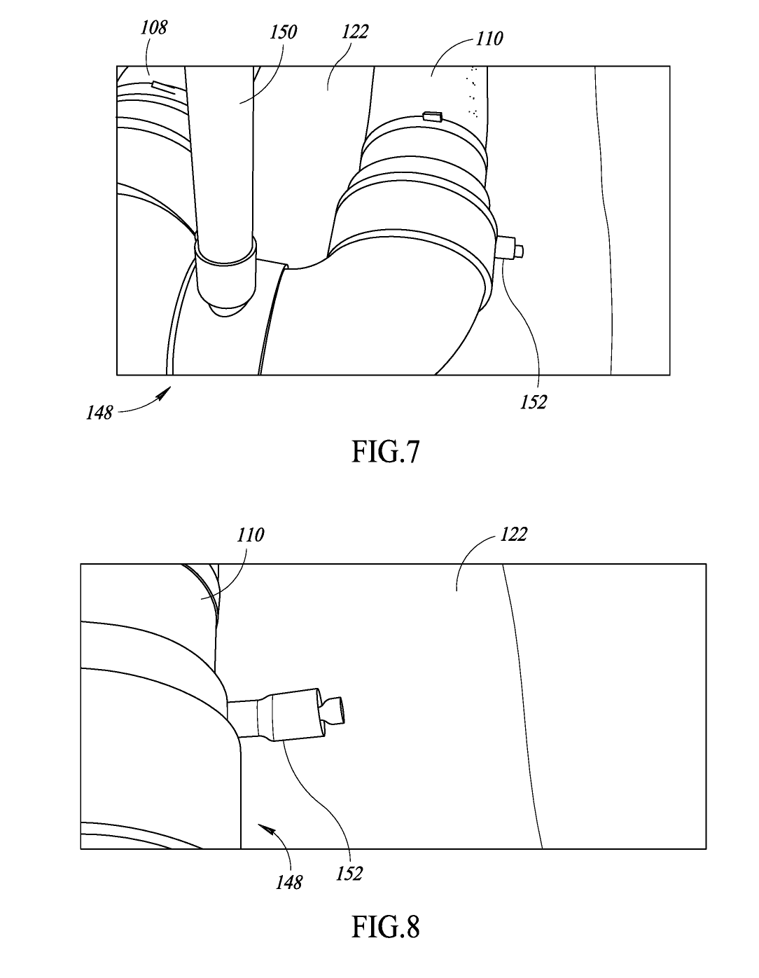

FIGS. 6-8 show the second corner 148 of the tube 120. The second corner 148 can have features similar to those described for the third corner 112, such as a vertical exhaust pipe 150 and a relief valve (not shown in FIGS. 6-8) similar to relief valve 136. FIGS. 7 and 8 show that the second corner 148 can also include a valve 152, which can allow CO₂ to be injected and dispersed into the tube 120. As one example, the valve 152 can include a small sintered metallic material, which can disperse CO₂ into micro-fine bubbles, which can be more readily absorbed into the fluid in the tube 120. While not shown in FIGS. 4-5, the third corner 112 can include a valve similar to the valve 152.

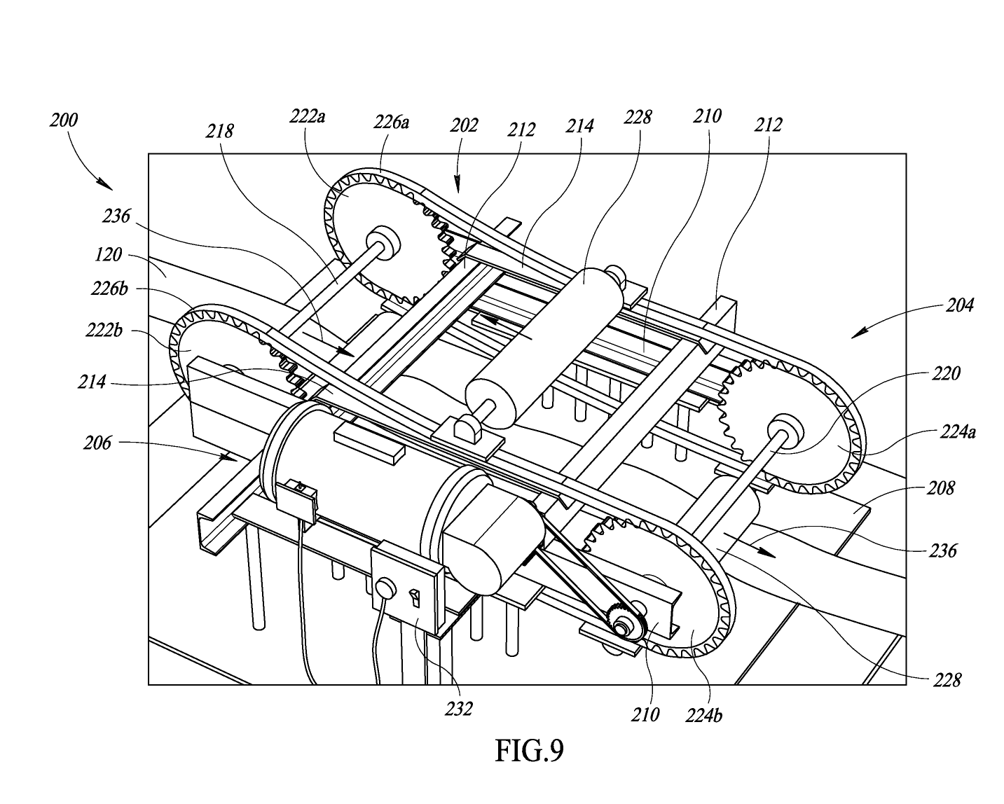

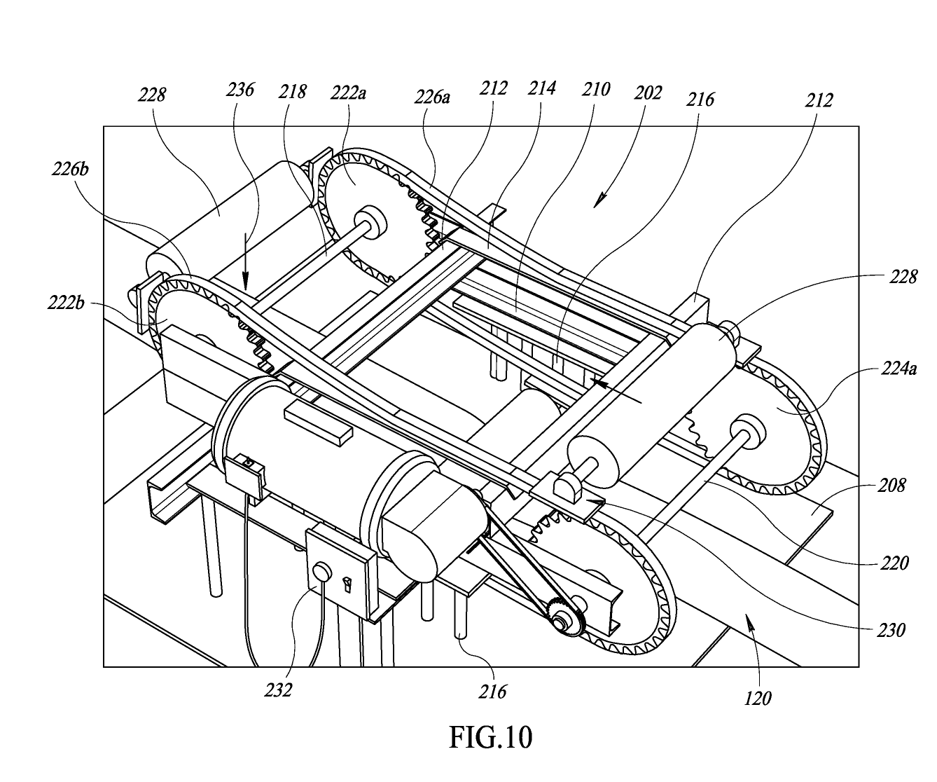

FIG. 9 shows the pump 200 inside the tent 102. The pump 200 is a peristaltic pump. The pump 200 includes a frame 202, a drive assembly 204, and a motor 206. The frame 202 can include at least one support or foundation plate 208, which can support other components of the pump 200. In some implementations, a single support plate 208 can be provided underneath the entire pump 200, while in other implementations, multiple smaller plates 208 can be provided underneath certain components of the pump 200. The frame 202 can also include longitudinal beams 210 oriented in generally the same direction as the tube 120. The beams 210 can be physically coupled to and raised off the support plate 208, such as by one or more vertical posts 216 (FIG. 10). The beams 210 illustrated in the figures are channel iron beams, but in alternative implementations, can be made from any suitable material and can have any suitable cross-sectional shape, for example, steel I-beams. In the implementation illustrated in the figures, the frame 202 includes a pair of beams 210, one positioned on either side of the tube 120. In alternative implementations, however, fewer or additional beams 210 can be used.

The frame 202 can also include one or more cross bars 212, which can be coupled to the beams 210 and span over and across the tube 120 and beams 210, and which can be oriented perpendicularly to the tube 120 and beams 210. In the implementation illustrated in the figures, the frame 202 can include two cross bars 212, each including a channel iron beam, but in alternative implementations, the frame can include fewer or additional cross bars 212, and cross bars of alternative material composition or cross-sectional shape, for example, steel I-beams. The cross bars 212 can couple the beams 210 to one another and thereby stabilize the frame 202. The frame 202 can also include one or more runners 214. In the implementation illustrated in the figures, the frame 202 includes two runners 214, each coupled to top surfaces of the pair of cross bars 212 and oriented in generally the same direction as the tube 120 and the beams 210. The runners 214 can be formed from steel plate material or other suitable materials.

FIG. 9 also shows that the drive assembly 204 includes a first, upstream axle 218 and a second, downstream axle 220. The first and second axles 218, 220 can be rotatably coupled to and supported by the beams 210, and can pass over and across the tube 120 in a direction oriented perpendicularly to the tube 120. The first axle 218 can be coupled to a first, upstream pair of sprockets 222a, 222b (collectively, 222), and the second axle 220 can be coupled to a second, downstream pair of sprockets 224a, 224b (collectively 224). The four sprockets 222, 224, can be coupled to the respective axles 218, 220, near the ends of the respective axles, such that the sprockets are above and spaced laterally apart from the tube 120. The sprockets 222, 224 can carry a pair of chains 226a, 226b (collectively, 226) meshed with the teeth of the sprockets 222, 224. For example, the upstream sprocket 222a and the downstream sprocket 224a can carry the chain 226a, which can be meshed with the teeth of the sprockets 222a and 224a. Similarly, the upstream sprocket 222b and the downstream sprocket 224b can carry the chain 226b, which can be meshed with the teeth of the sprockets 222b and 224b.

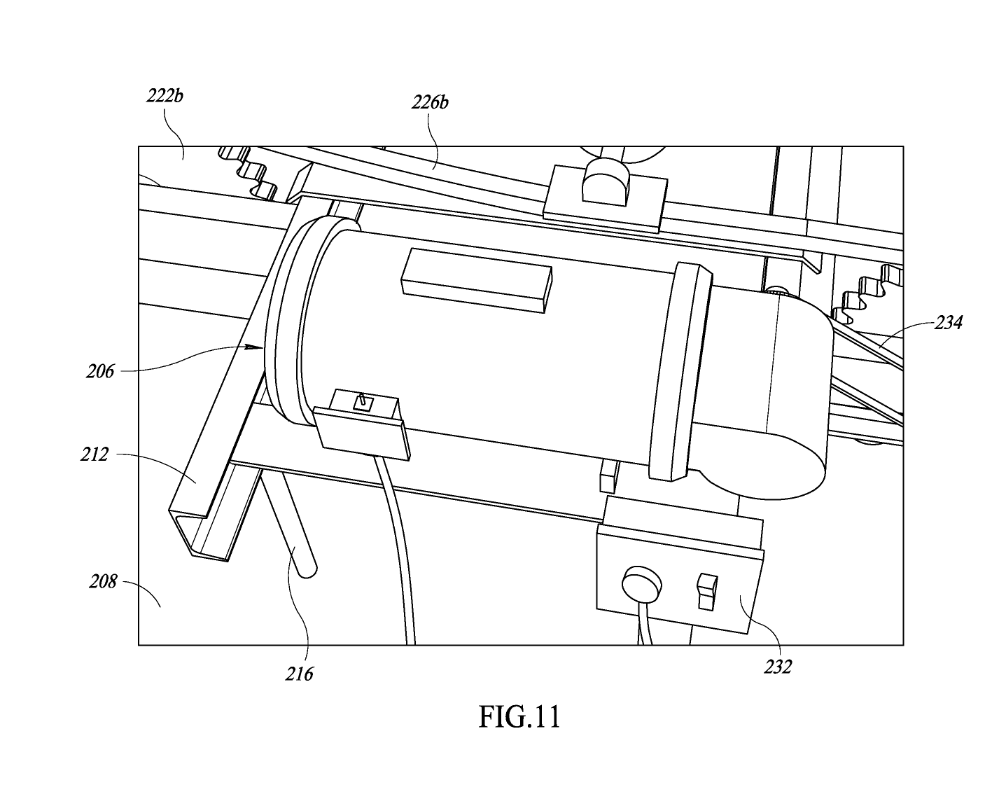

The chains 226 can carry a plurality of rollers 228 mounted to the chains 226 on mounting elements 230 (FIG. 10). The mounting elements 230 can be fixed to the chains 226 and can include cylindrical bearings that allow the rollers 228 to rotate freely about their longitudinal axes, with respect to the mounting elements 230. In some implementations, the chains 226 are supported by the runners 214 as they travel between the first and second sprockets 222, 224. In some implementations, the rollers can be formed from hollow or solid cylindrical elements such as a hollow steel cylinder or a solid concrete cylinder. FIG. 11 shows that the motor 206 is coupled to and drives the second axle 220 and thereby drives the pump 200, such as by a drive chain 234. The motor 206 can be controlled to drive the second axle 220 and the pump 200, such as by a switch 232. In alternative implementations, the motor 206 can drive the pump 200 via the first axle 218 or both the first and second axles 218, 220, and the motor 206 can be controlled in alternative ways, such as by a dial that controls the speed at which the motor 206 drives the pump, by remote control, etc.

When running, the motor 206 turns the drive chain 234, which turns the second axle 220, which turns the second sprockets 224, causing the chains 226 and thus the rollers 228 to rotate around the first and second sprockets 222, 224. As the rollers 228 rotate around the sprockets 222, 224, the rollers 228 follow a path which brings them into contact with, or drives them into the tube 120, compressing (or pinching or occluding) it to some degree, as shown for example in FIG. 9. In various implementations, the rollers follow a path which causes them to compress the tube 120 such that the tube 120 has a height less than half, less than a quarter, less than 10%, or less than 5% of its original diameter. In some implementations, the rollers 228 follow a path which causes them to compress the tube 120 such that the tube 120 is sealed at the location of contact with the roller 228, while in other implementations, the rollers 228 follow a path which causes them to compress the tube 120 but not to the extent that the tube 120 is sealed at the location of contact with the roller 228.

In the implementation shown in the figures, the rollers 228 follow a path indicated by arrows 236 (FIGS. 9 and 10). Specifically, the rollers 228 move upward away from the tube 120 as they move over the downstream sprockets 224, over the tube 120 toward the upstream sprockets 222, downward toward the tube 120 as they move over the upstream sprockets 222, until they compress the tube 120, and then toward the downstream sprockets 224 as they maintain compression of the tube 120. As a roller 228 moves from the upstream sprockets 222 toward the downstream sprockets 224, the location at which the roller 228 compresses the tube 120 translates across the tube 120 in the downstream direction. This forces the fluid in the tube to move downstream, effecting the pumping action of the pump 200. As the rollers 228 move across the tube 120 in this manner, the rollers 228 can roll on their bearings, rolling across the tube, thereby reducing the friction between the rollers 228 and the tube 120.

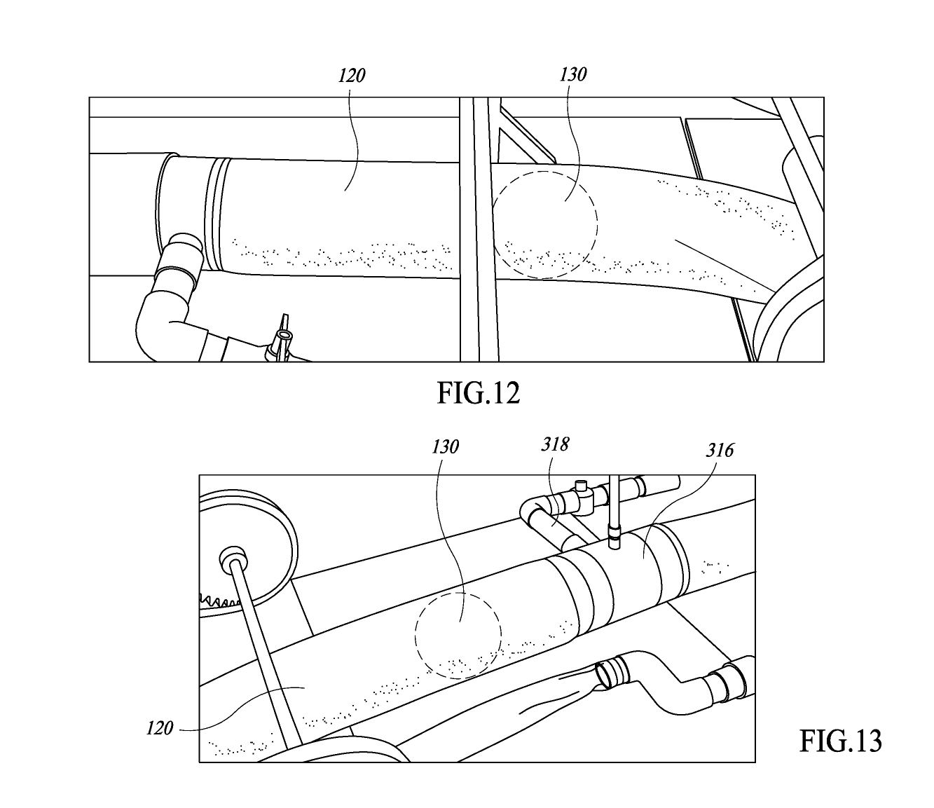

FIGS. 12 and 13 show the pumping action of the pump 200, which forces the ball 130 to move in the downstream direction, i.e., from the position shown in FIG. 12 (where the ball 130 is approaching the pump 200), to the position shown in FIG. 13 (where the ball 130 is leaving the pump 200).

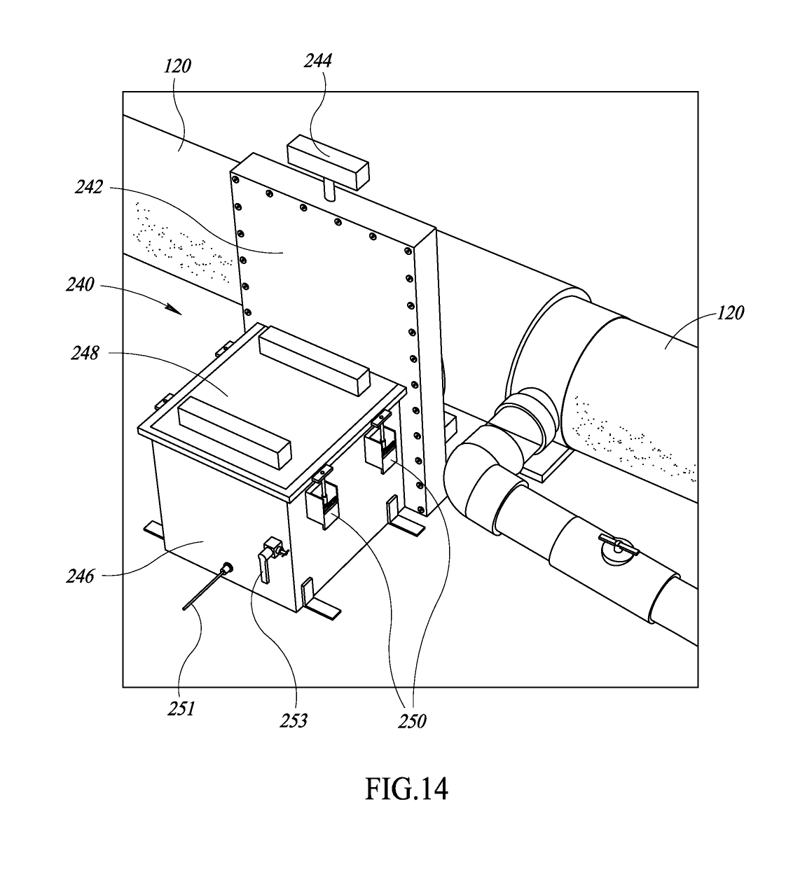

FIGS. 14-16 show a ball trap assembly 240 that allows the timing of the balls 130 passing through the tube 120 to be coordinated or synchronized with the timing of the rollers 228 passing over the tube 120, and that allows balls 130 to be added to and removed from the tube 120. The ball trap assembly 240 can include a housing 246 having a lid 248, which can be coupled to the housing 246 by latches 250, such as on a first side of the housing 246 (not shown) and on a second side of the housing 246 opposite to the first side of the housing 246. The ball trap assembly 240 also includes a valve 253 such as a needle valve, which can be coupled to an air compressor (e.g., through a HEPA filter) and can allow an operator to selectively introduce a gas (e.g., air) into, or remove such a gas from, the interior of the housing 246. The ball trap assembly 240 also includes a valve housing 242 which can include a valve element (not illustrated) such as a plate of material which can be actuated to move vertically within the valve housing 242 by a handle 244.

An interior of the housing 246 can be fluidically coupled to the interior passage 121 of the tube 120 via an orifice through the valve housing 242. The handle 244 can be moved from a first, lower position (as shown in FIG. 14) to a second, higher position (not illustrated) to actuate the valve element to move within the valve housing 242 from a first, lower, closed position, in which the valve element covers the orifice and fluidically seals the interior of the housing 246 from the interior passage 121 of the tube 120, to a second, higher, open position, in which the valve element does not cover the orifice and does not seal the interior of the housing 246 from the interior passage 121 of the tube 120. The orifice can have dimensions such as a diameter sufficient to allow the balls 130 to pass into and out of the tube 120.

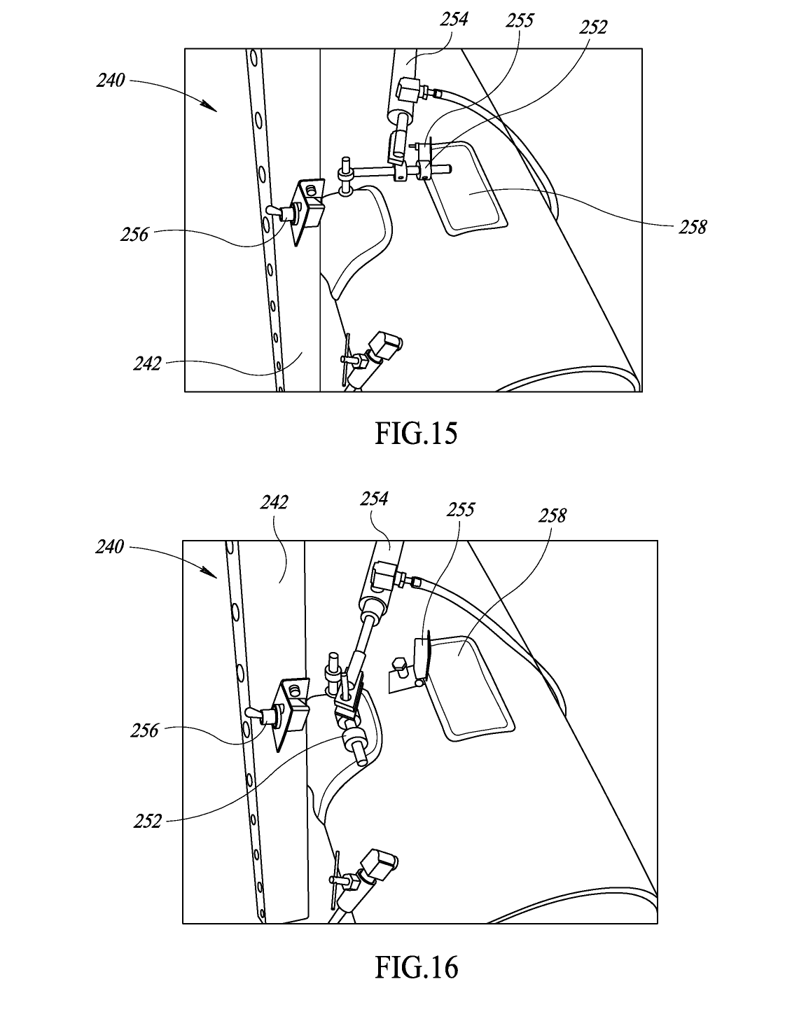

FIGS. 15 and 16 show that the ball trap assembly 240 also includes a lever 252 which can be rigidly coupled to and thereby control actuation of a mechanical retention element or gate (not illustrated) within the tube 120. The gate can be positioned within the tube 120 upstream or downstream of the valve housing 242. In some implementations, the gate is positioned downstream of the valve housing 242 so that balls 130 introduced into the tube 120, as described above, are initially retained in place by the gate. The lever 252 can be actuated to move from a first, open position (shown in FIG. 16) to a second, closed position (shown in FIG. 15) to actuate the gate to move within the tube 120 from a first, open position, in which the balls 130 can pass by the gate through the tube 120, to a second, closed position, in which the gate retains balls 130 as they pass through the tube 120. The lever 252 can be controlled to move between the first and second positions by a piston 254, which can be a pneumatically powered piston 254. In some implementations, the tube 120 can include a window 258, which can allow a person to see whether a ball 130 is being retained by the gate within the tube 120.

In some implementations, three switches coupled to one another in series can control operation of the piston 254 such that the piston 254 can be actuated when all three switches are closed to form a closed circuit. For example, a first manual switch 256 can allow an operator to manually prevent actuation of the piston 254 by opening the first manual switch 256. A second switch 255 can include a mechanical protrusion or detent which is depressed by the lever 252 until a ball 130 travelling through the tube 120 collides with the gate, causing the lever 252 to move with respect to the mechanical detent of the second switch 255, thereby closing the second switch 255.

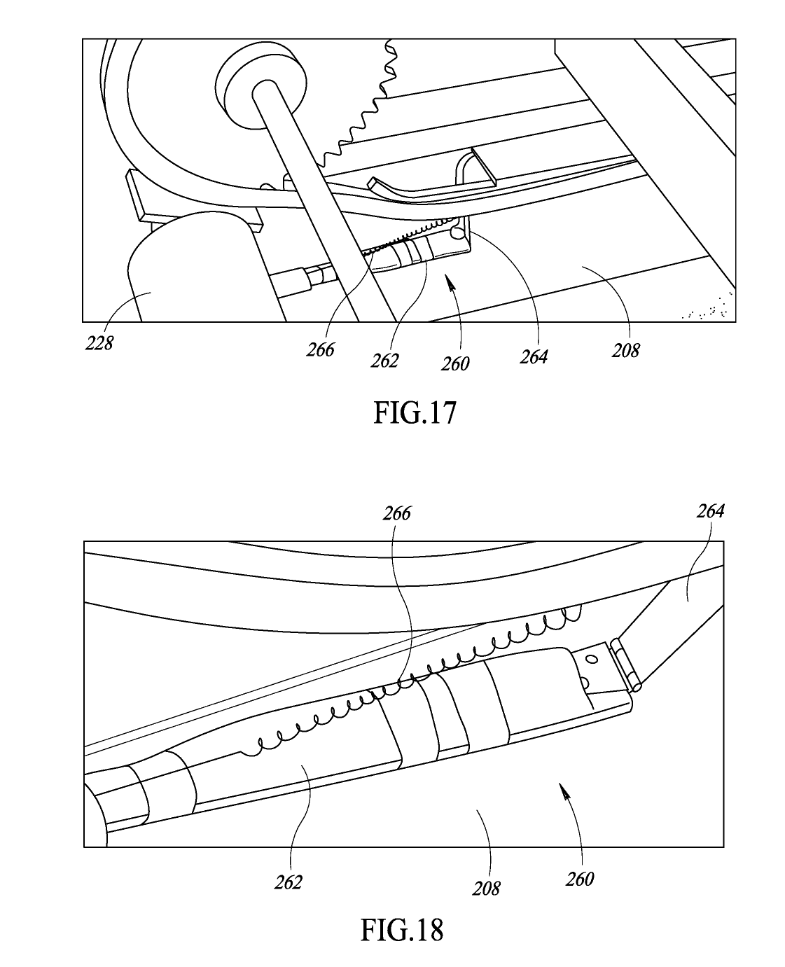

A third switch can be an automated switch 260 that can synchronize actuation of the piston 254 with the movement of the rollers 228. For example, FIGS. 17-18 show that an automated switch 260 can include a base plate 262, which can be mounted or coupled to the support plate 208 (e.g., such as by magnets, which can allow simple movement of the automated switch 260 across the support plate 208), a rotatable arm 264, which can be rotatably coupled to the base plate 262, such as by a hinge, and a spring 266. The rotatable arm 264 can be rotated with respect to the base plate 262 from a first, open position, as shown in FIG. 17, to a second, closed position, as shown in FIG. 18. A mercury tilt switch, which can open and close based on its orientation with respect to gravity, can be coupled to the arm 264 so as to effect the opening and closing of the automated switch 260 as the arm 264 rotates. The mercury tilt switch can be coupled to the arm 264 so as to be open when the arm 264 is in its open position and closed when the arm 264 is in its closed position.

The spring 266 can be coupled to the base plate 262 and the arm 264 so as to bias the arm 264 to rotate toward the first, open position, as shown in FIG. 17. In one implementation, the automated switch 260 can be positioned such that as the roller 228 moves past the automated switch 260, the roller 228 catches the rotatable arm and moves it from the first, open position, to the second, closed position, as shown in FIG. 18. Once the roller 228 has passed the automated switch 260, the spring 266 can cause the arm 264 to return to the first, open position. In some implementations, the spring 266 is not used, and the arm 264 can be balanced to return on its own to the first, open position once the roller 228 has passed the automated switch 260.

The automated switch 260 can thus control the timing of the balls 130 passing through the tube 120, such as to coordinate or synchronize the timing of the balls 130 with the timing of the rollers 228, and the automated switch 260 and the ball trap assembly 240 can thus be referred to together as a synchronizer. This coordination or synchronization can prevent the balls 130 from interfering with the rollers 228, and thereby prevent damage occurring to the pump 200. The automated switch 260 can thus regulate the passage of the balls 130 though the system, whether the balls 130 are returning to the pump 200 after completing a circuit through the tube 120, or whether the balls 130 have been newly added to the tube 120 at the ball trap assembly 240.

To selectively insert a ball 130 into the tube 120, the lid 248 can be latched onto the housing 246 to form a fluid-tight seal enclosing the interior of the housing 246. The handle 244 can be used to move the valve element toward the upper, open position (e.g., by about one inch), and air can be pumped into the housing 246 through the valve 253, so as to force a portion of the liquid in the housing 246 into the tube 120 and thereby lower the water level in the housing 246. The handle 244 can then be used to move the valve element to the lower, closed position. The latches 250 can be released, the lid 248 can be lifted off the top of the housing 246, and a ball 130 can be inserted into the housing 246, with the fluid remaining in the housing 246 surrounding the ball 130. In some implementations, the pump 200 can either be turned off or be left running, and the manual switch 256 can be opened to prevent the gate being opened during the selective insertion of the ball 130 into the tube 120. The lid 246 can be latched onto the housing 246 once again, the handle 244 can then be used to move the valve element to the upper, open position, and the ball 130 can be moved into the tube 120. In some implementations, the ball can be pushed from the housing 246, through the valve housing 242, and into the tube 120 by a rod 251 coupled to a ball carriage within the housing 246. The handle 244 can then be used to move the valve element toward the lower, closed position, e.g., so that it is within about one inch of the lower, closed position, the pump 200 can then be turned on if it was turned off, and the manual switch 256 can be closed to allow the gate to be opened. The ball carriage can remain within the tube 120 during operation.

To selectively remove a ball 130 from the tube 120, a process similar to that for inserting a ball 130, but in reverse, can be used. The handle 244 can be used to move the valve element to its upper, open position, the ball carriage can be positioned within the tube 120, and once a ball 130 has arrived at the gate (e.g., as viewed through the window 258) and ball carriage, the pump 200 can either be turned off or be left running, and the manual switch 256 can be opened to prevent the gate being opened during the selective removal of the ball 130 from the tube 120. The ball 130 can then be moved from the tube 120, through the valve housing 242, and into the housing 246. In some implementations, the ball can be pulled from the tube 120, through the valve housing 242, and into the housing 246 using the rod 251 coupled to the ball carriage within the housing 246. The handle 244 can then be used to move the valve element toward its lower, closed position (e.g., such that it is about one inch from the lower, closed position). Air can be pumped into the housing 246 through the valve 253, so as to force a portion of the liquid in the housing 246 into the tube 120 and thereby lower the water level in the housing 246. The handle 244 can then be used to move the valve element to the lower, closed position. The latches 250 can be released, the lid 248 lifted off the top of the housing 246, and the ball 130 can be removed from the housing 246. The pump 200 can then be turned on if it was turned off, and the manual switch 256 can be closed to allow the gate to be opened.

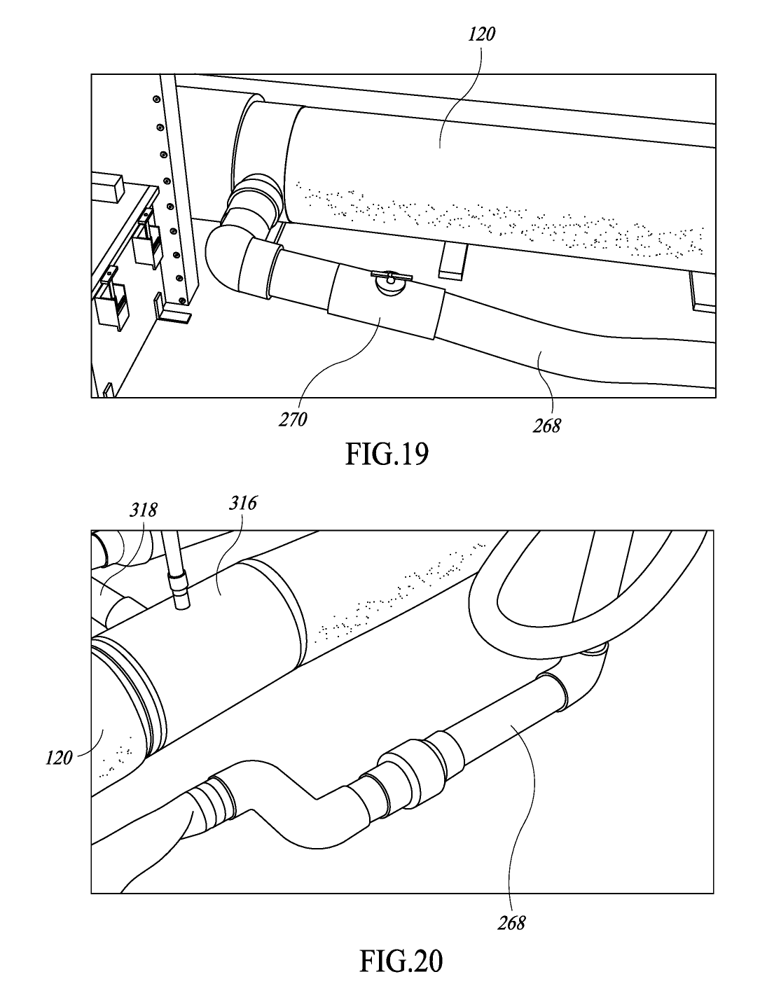

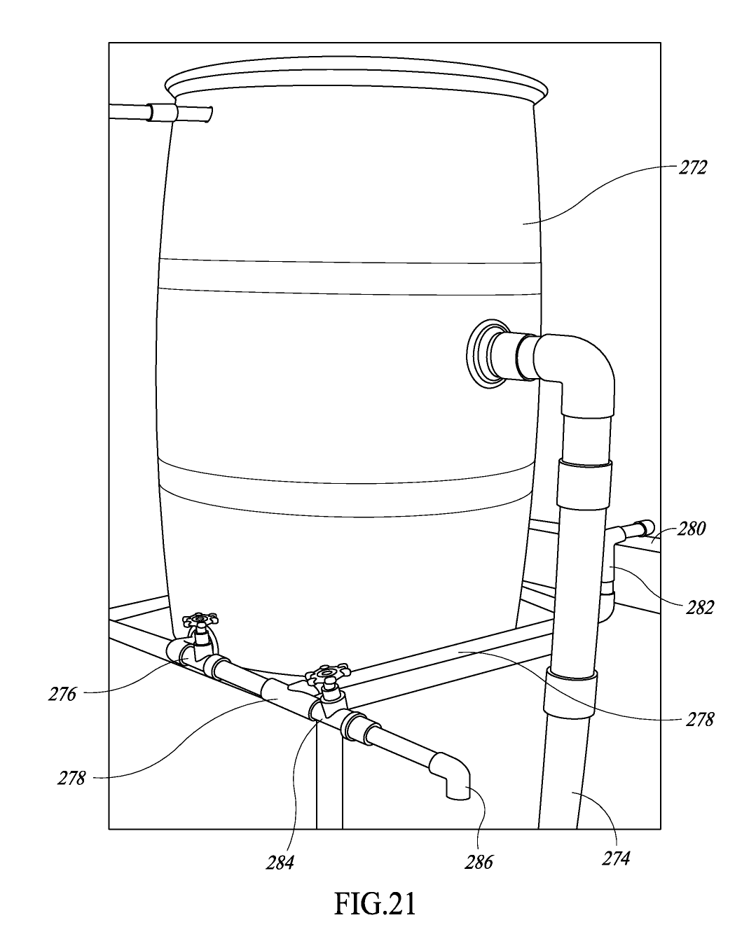

FIGS. 19-21 show that the system 100 can include an algae harvesting system including a pipe 268 fluidically coupled to the tube 120 via a valve 270. The valve 270 can remain closed to prevent fluid and algae from escaping the tube 120, and can be opened to allow fluid and algae to flow along the pipe 268 to a holding tank 272. When the valve 270 is opened, the fluid and algae can flow through the harvesting pipe 268, into the holding tank 272. In the illustrated implementation, the fluid and algae can flow upward through a vertical portion 274 of the harvesting pipe 268 to the holding tank 272, such as due to the pressure maintained in the tube 120 by the pump 200.

The harvesting system can also include a valve 276 fluidically coupled to the holding tank 272, such as near the bottom of the holding tank 272. When the valve 276 is opened, fluid and algae held in the holding tank 272 can flow through the valve 276 into a pipe 278, and along the pipe 278 to an algae-harvesting apparatus 280. In some implementations, the fluid and algae can flow upward through a vertical portion 282 of the pipe 278 into the algae-harvesting apparatus 280, such as due to a pressure maintained in the pipe 278 by a level of the fluid in the holding tank 272. The pipe 278 can be fluidically coupled to a release valve 284. When the release valve 284 is opened, fluid and algae can flow through the valve 284 into a release pipe 286 and out of the harvesting system. Thus, the release valve 284 and release pipe 286 can allow the holding tank 272 to be drained.

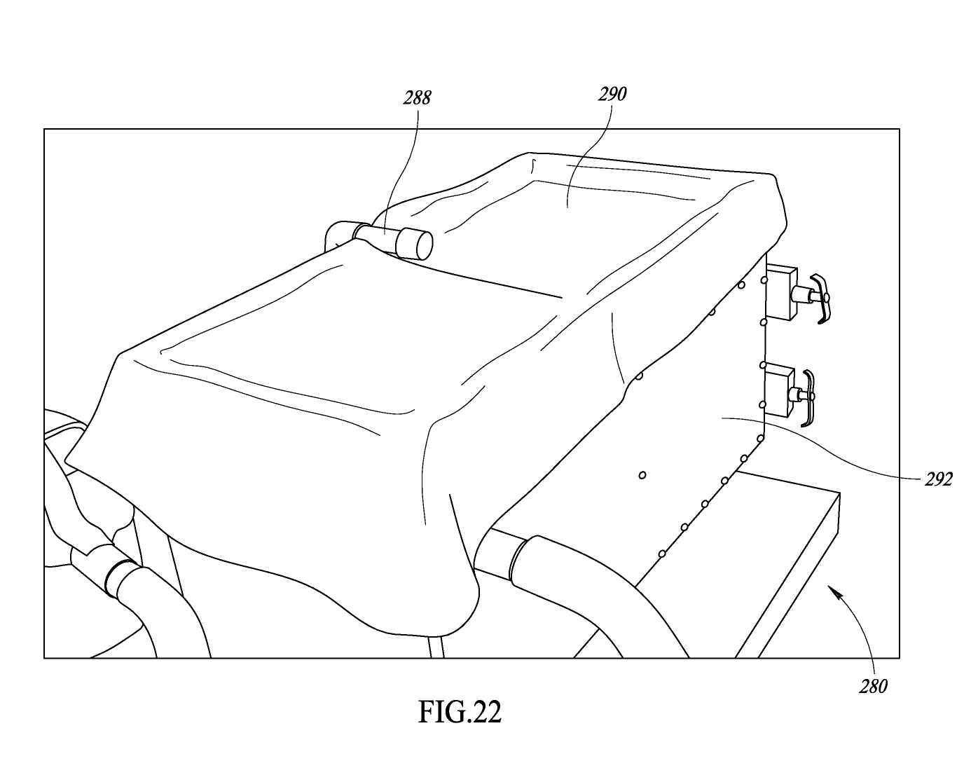

FIGS. 22-25 show the harvesting apparatus 280 in greater detail. FIG. 22 shows that the pipe 278 can terminate at a spout 288, which can be positioned over a filter 290 covering a tank 292, such that fluid and algae can flow through the pipe 278, out of the spout 288, and onto the filter 290. The filter 290 can allow the fluid to pass through, while preventing the algae from passing through into the tank 292. To harvest algae from the system, fluid and algae can be allowed to flow out of the spout 288, and onto the filter 290, where the algae can be separated from or substantially separated from the fluid. Once the algae has been separated from the fluid, it can be packaged, such as in sterile containers, for transportation to other locations, for storage, for sale, or for other uses.

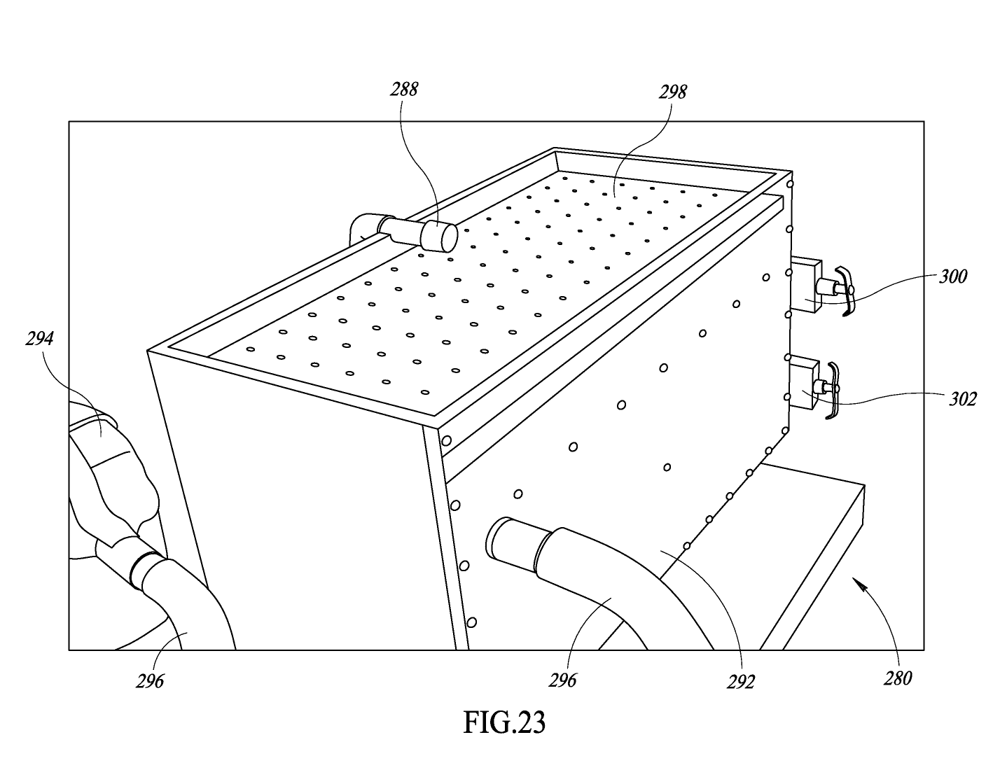

FIGS. 23-25 show additional details regarding the tank 292 and its operation. FIG. 23 shows that the harvesting apparatus can also include a perforated plate 298 situated underneath the filter 290 and on top of the tank 292, and an air compressor 294 fluidically coupled to the tank 292 via a pipe 296. The air compressor 294 can be used to reduce the air pressure in a bottom portion of the tank 292, thus drawing the fluid through the filter 290 and into the tank 292, to separate the fluid from the algae. The harvesting system can also include a first valve 300 which can fluidically couple a top portion (described in greater detail below) of the tank 292 to the bottom portion of the tank 292, and a second valve 302 which can fluidically couple the bottom portion of the tank 292 to a drain pipe, such as, in some implementations, the hose 128. In some implementations, the second valve 302 can be opened to allow fluid to simply drain out of the drain pipe. In other implementations, the second valve 302 can be opened and the air compressor 294 can be used to increase the air pressure in the bottom portion of the tank 292, thus forcing the fluid through the second valve 302, into, through, and out of the drain pipe. In some implementations, the drain pipe can drain the excess fluid back into the holding tank 272 or back into the tube 120.

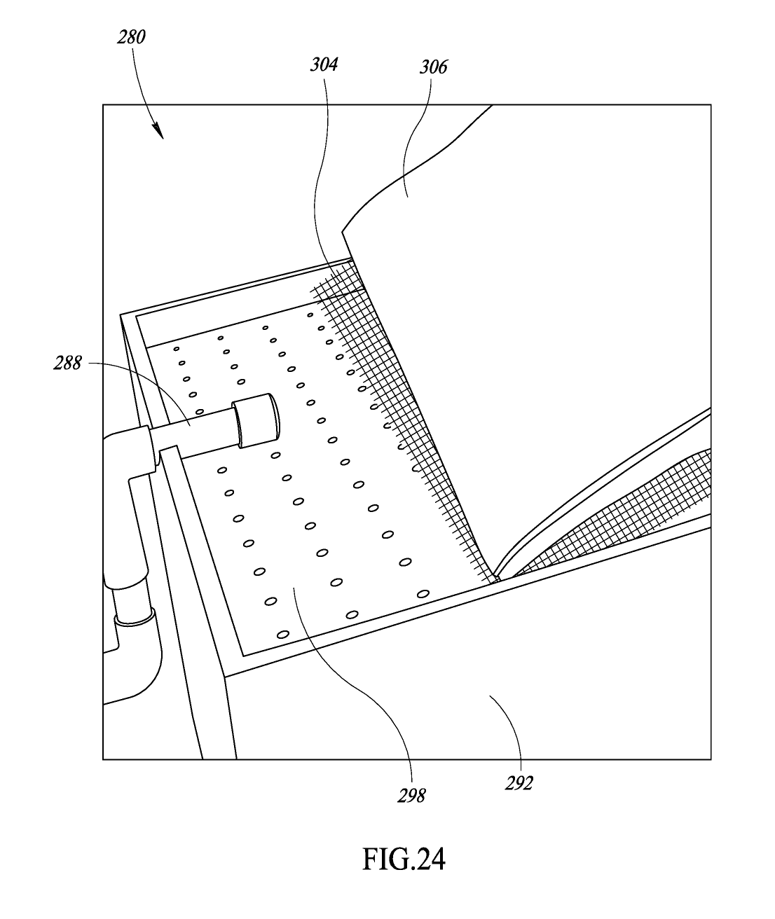

FIG. 24 shows that the harvesting apparatus can also include a mesh sheet 304 which can be positioned between the filter 290 and the perforated plate 298 during operation of the harvesting apparatus, such as to prevent the filter being pulled into the perforations of the perforated plate 298, and to separate the fluid, algae, and filter 290 from the perforated plate 298 when the air compressor 294 is used to draw fluid through the filter 290. The harvesting apparatus can also include one or more sheets of protective material 306 which can be used to cover the perforated plate 298, mesh sheet 304, or the filter 290 when the harvesting apparatus is not in use, such as to protect these elements.

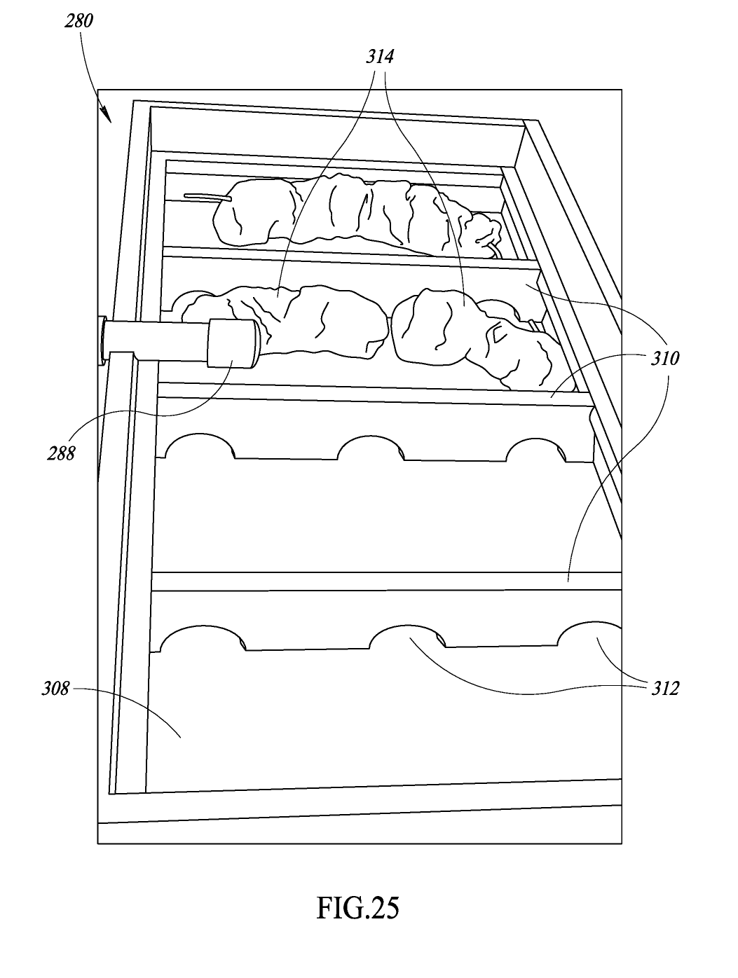

FIG. 25 shows the top portion of the tank 292 under the perforated plate 298 in greater detail. FIG. 25 shows that the top portion of the tank 292 can include a bottom plate 308 which can also form a top plate 308 of the bottom portion of the tank 292 and thereby separate the top portion of the tank 292 from the bottom portion of the tank 292. The plate 308 can be sloped toward the end of the tank including the first and second valves 300, 302, so that fluid drawn through the filter 290 drains toward the first valve 300. The upper portion of the tank 292 can also include a plurality of vertical plates 310 having a bottom end portion in contact with the plate 308 and extending from the plate 308 toward the top of the tank 292. Each of the vertical plates 310 can have a plurality of semi-circular openings 312 formed at its bottom end portion. The vertical plates 310 and their openings 312 can help to condense airborne fluid droplets drawn through the filter 290, to help prevent their being drawn into the air compressor 294 and interfering with its operation. The top portion of the tank 292 can also include condensing elements 314 such as steel wool, positioned between the vertical plates 310, to further help condense airborne fluid droplets and prevent their being drawn into the air compressor 294.

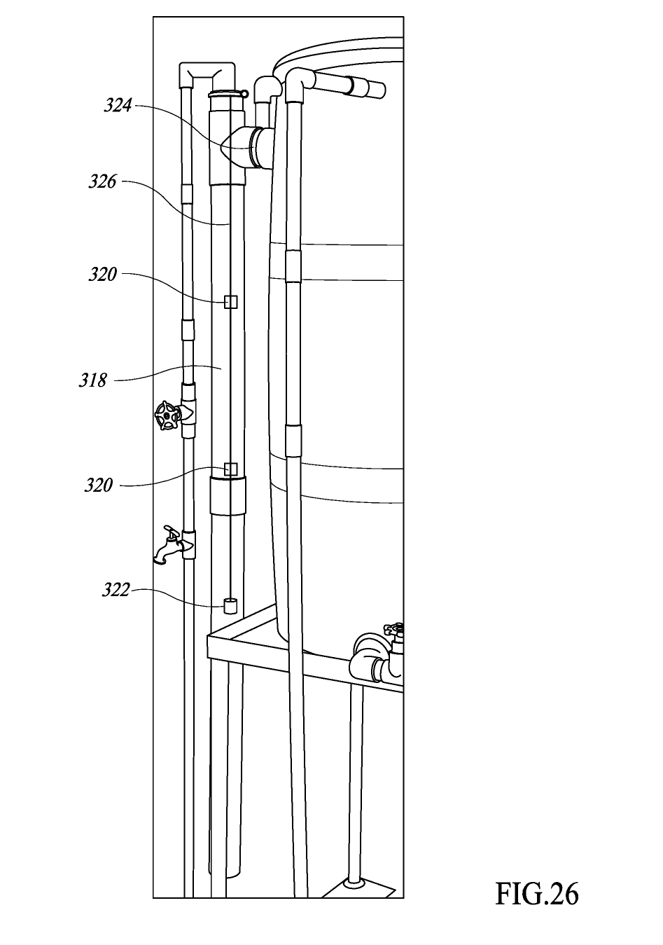

FIG. 26 shows a fluid pressure measurement apparatus that allows the pressure of the fluid in the tube 120 to be measured. FIG. 26 illustrates a vertical portion of a measurement pipe 318, which can be fluidically coupled to the tube 120, such as at a location 316 (FIGS. 13 and 20) so that a height of the fluid in the vertical portion of the pipe 318 is indicative of the pressure of the fluid in the tube 120. A first weight (not illustrated) lighter than the fluid in the tube 120, such as a cork, can be positioned within the vertical portion of the pipe 318 to float on top of the fluid in the pipe 318. The first weight can be coupled to a cable 326, which can run upward through the pipe 318, over the top of the pipe 318, and down the side of the pipe 318, where it can be coupled to a second weight 322, which can be lighter than the first weight such that the second weight 322 moves upward as the water level in the vertical portion of the pipe 318 moves downward and such that the second weight 322 moves downward as the water level in the vertical portion of the pipe 318 moves upward. The vertical portion of the pipe 318 can include markings 320 spaced at regular known intervals, such as at one foot increments, and thus by comparing the location of the second weight 322 to the markings 320, the fluid pressure in the tube 120 can be determined.

A relief pipe 324 can fluidically couple the vertical portion of the pipe 318 to the holding tank 272, such as by coupling a top portion of the vertical portion of the pipe 318 to the holding tank 272. In the event the pressure in the tube 120 increases to undesirably high levels, the fluid can escape out of the tube 120, up the vertical portion of the pipe 318, through the relief pipe 324, and into the holding tank 272, thereby relieving the pressure in the tube 120. By selecting the height at which the relief pipe 324 is fluidically coupled to the vertical portion of the pipe 318, a maximum tube 120 pressure can be established. In some implementations, algae can be seeded into the system 100 through the vertical portion of the pipe 318, such as by simply dropping it into the open top end of the vertical portion of the pipe 318. Additional materials, such as water, nutrients (e.g., phosphorus or potassium), or inoculants, can be introduced into the system 100 in a similar manner.

While the pump 200 can be used to increase the pressure in the tube 120 to any desirable pressure, it has been found that using the system 100 on relatively flat ground and maintaining relatively low fluid pressures in the tube 120 allows desirable algae growth. In the illustrated implementation, the tube 120 is positioned on nearly level ground, has a diameter of about 8 inches, has a length of 250 feet, can be made from a flexible, resilient material having a thickness of 4 mil or 6 mil, and can carry water containing algae. It has been found that in this implementation, the pump 200 can continuously pump the fluid (which amounts to approximately 650 gallons of water) through the tube 120 at a speed of at least 30 feet per minute while maintaining a pressure of about 2.5-3.0 feet of pressure head, and can maintain a pressure of at least 4.0 feet of pressure head. In alternative implementations, the system 100 can include a tube 120 having a length in excess of 1000 feet.

In some implementations, the height of the rollers 228 with respect to the ground and the tube 120 can be adjustable. For example, the beams 210 can be adjustably coupled to the vertical posts 216 such that the frame 202 can be raised and lowered. In such implementations, the frame 202 can be raised to decrease the pressure created in the tube 120 by the pump 200 and to decrease the speed at which the pump 200 pumps fluid through the tube 120, or the frame 202 can be lowered to increase the pressure created in the tube 120 by the pump 200 and to increase the speed at which the pump 200 pumps fluid through the tube 120.

In some implementations, the speed at which the rollers 228 rotate around the frame 202 can be adjustable. For example, the motor 206 can be adjustable to drive the second axle 220 at various speeds, as desired. The speed of the rollers can be increased to increase the speed at which fluid flows through the tube 120, or the speed of the rollers can be decreased to decrease the speed at which fluid flows through the tube 120.

The system 100 allows algae to be grown in a closed system, which can provide greater control over the algae growth process and can reduce environmental impacts of non-closed algae growth systems. Because it is a closed system, the system 100 can also be particularly advantageous in low-water environments, because very little water is consumed by the system 100. In particular, because the system 100 works well on flat ground surfaces and is particularly advantageous in dry environments, dried lake beds provide a very suitable environment for implementing the system 100. The system 100 is also advantageous due to its use of a peristaltic pump, which is less destructive to the algae, and which allows the balls 130 to continuously flow through the tube 120.

U.S. provisional patent application No. 62/093,304, filed Dec. 17, 2014, to which this application claims priority, is hereby incorporated herein by reference in its entirety. The various embodiments described above can be combined and modified to provide further embodiments. Those of skill in the art will recognize that many of the methods set out herein may employ additional acts, may omit some acts, and/or may execute acts in a different order than specified.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A system, comprising:
a looped tube that holds water, algae, and a ball;
a peristaltic pump positioned to pump the water, the algae, and the ball through the looped tube; and
a synchronizer coupled to synchronize pumping action of the peristaltic pump with movement of the ball through the looped tube.

2. The system of claim 1 wherein the looped tube lies within a horizontal plane.

3. The system of claim 1 wherein the looped tube lies on a dried lake bed.

4. The system of claim 1 wherein the ball is a first ball that is heavier than water and the looped tube holds a second ball that is lighter than water.

5. The system of claim 1 wherein the ball has a diameter that is within two inches of an inside diameter of the looped tube.

6. The system of claim 1, further comprising a reflective tarp positioned under the looped tube.

7. The system of claim 1, further comprising a vertical exhaust pipe having a bottom end coupled to the looped tube and a top end coupled to a relief valve.

8. The system of claim 1, further comprising an input valve including a sintered metal.

9. A method, comprising:
positioning water, algae, and a ball within a looped tube;
driving a roller of a peristaltic pump to compress the looped tube;
translating the roller across the looped tube, thereby pumping the water, the algae, and the ball through the looped tube; and
synchronizing the driving of the roller of the peristaltic pump to compress the looped tube with a passage of the ball through the looped tube.

10. The method of claim 9 wherein positioning the ball within the looped tube includes:
closing a valve between a housing and the looped tube;
positioning the ball within the housing;
sealing the housing;
opening the valve; and
moving the ball from the housing into the looped tube.

11. The method of claim 9, further comprising removing the ball from the looped tube, the removing comprising:
retaining the ball in place within the looped tube;
opening a valve between a housing and the looped tube;
moving the ball from the looped tube into the housing;
closing the valve;
opening the housing; and
removing the ball from the housing.

12. A system, comprising:
a looped tube that has a passage having a first diameter to contain water, algae, and a ball having a second diameter; and
a valve that has an orifice fluidically coupled to the looped tube, wherein the orifice has a third diameter greater than the second diameter such that a difference between the first diameter and the third diameter is less than two inches.

13. A method, comprising:
using a peristaltic pump to pump water and algae through a looped tube;
drawing a portion of the water and algae out of the looped tube;
positioning a filter on a perforated plate;
pouring the portion of the water and algae onto a first surface of the filter; and
reducing an air pressure on a second surface of the filter opposite the first surface to draw the water through the filter.

14. The method of claim 13 wherein drawing the portion of the water and algae out of the looped tube includes drawing the portion of the water and algae into a holding tank.

15. The method of claim 14 wherein pouring the portion of the water and algae onto the first surface of the filter includes pouring the first portion of the water and algae from the holding tank.

16. The method of claim 15, further comprising measuring a fluid pressure within the looped tube by observing a water level in a vertical portion of a pipe coupled to the looped tube.

17. The method of claim 16, further comprising, when the fluid pressure within the looped tube exceeds a threshold fluid pressure, relieving the fluid pressure within the looped tube by allowing the water and algae to flow from the looped tube, through the vertical portion of the pipe, into the holding tank.
